(19) United States
(12) Patent Application Publication
(10) Pub. No.: US 2008/0293130 A1
(43) Pub. Date: Nov. 27, 2008

(54) BIOCHIP

(75) Inventors: Yoshiyuki Miyoshi, Minami-Ashigara-shi (JP); Takayuki Fujiwara, Minami-Ashigara-shi (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM CORPORATION, Tokyo (JP)

(21) Appl. No.: 12/044,624

(22) Filed: Mar. 7, 2008

(30) Foreign Application Priority Data
Mar. 9, 2007 (JP) 2007-060722

(51) Int. Cl. C12M 1/34

(52) U.S. Cl. 435/288.7

(57) ABSTRACT

A biochip that can make optical measurements without being affected by background fluorescence etc., originating from the outside environment, wherein the surface of the substrate of the biochip can be chemically treated by a simple procedure. The biochip has at least a nontransparent resin substrate, an immobilizing layer formed on a part of the non-transparent resin substrate, and an organic molecule immobilized on the immobilizing layer, and detects an analyte labeled with fluorescence or luminescence in a sample, from the intensity of the fluorescence or luminescence.

### BACKGROUND OF THE INVENTION

1. Field of the Invention

The present invention relates to a biochip, more specifically, to a biochip that detects an analyte labeled with fluorescence or luminescence in a sample, from the intensity of the fluorescence or luminescence.

2. Description of the Related Art

In recent years, there has been increasing attention on the so-called biochips in which the entire series of processes needed to detect an analyte in a sample, to gain information about the sample, is performed in a tiny space of the order of a micrometer.

Optical and electrical detection methods are generally known as methods of detecting an analyte with a biochip. Especially, the method in which the analyte in the sample is labeled with fluorescence or luminescence, and the intensity of the fluorescence or luminescence is measured, is often used as an optical detection method because the method is comparatively simple and quantitative.

In the aforesaid optical detection, the intensity of the fluorescence or luminescence is measured. Therefore, for removing noise, the effect of the background fluorescence arising from the substrate, external environment, or the shape of the substrate (its edges, for instance) need to be suppressed to the extent possible.

Therefore, the use of a comparatively inexpensive and easy-to-mold resin as a substrate material for the biochip, and making the resin substrate non-transparent to suppress background fluorescence have been suggested, for instance, in the Japanese Patent Application Laid-Open No. 2003-130874 and Japanese Patent Application Laid-Open No. 2005-189050.

Also, the use of a glass (especially quartz glass) as a substrate material for the biochip has been suggested in the Japanese Patent Application Laid-Open No. 2006-126179. The reason is that glass used as the substrate material can suppress the fluorescence originating from the substrate itself, such as seen with resin substrates.

Apart from the issue of noise elimination, when a micro component in the sample is to be detected with high sensitivity by a biochip, the surface in different areas of the substrate need to be functionally modified (for specific adsorption of a biomolecule or for prevention of nonspecific adsorption of biomolecules) so as to capture the analyte only in the detection area.

In the aforesaid resin substrate, the surface is normally inactive, and this makes it difficult to give a chemical treatment such as silane coupling, for functional modification of the surface. For this purpose, the Japanese Patent Application Laid-Open No. 2005-121443 suggests an oxidation/reduction treatment of the surface of the resin substrate, followed by a chemical treatment like silane coupling.

Contrary to this, the surface of a glass substrate has a large number of OH groups, and has the characteristic of being easy to treat chemically, such as by the aforesaid silane coupling, and being easier than resin substrates to create a good quality functional surface thereon. However, glass substrates have the drawbacks of poorer moldability, and higher material cost compared to a resin substrate.

### SUMMARY OF THE INVENTION

The purpose of the present invention is to provide a biochip, which can be made with low cost of materials, molding, and other processes, and allows optical measurements unaffected by background fluorescence originating from the substrate material and shape, and the outside environment; and furthermore, the surface of the substrate of which can be easy to treat chemically.

The biochip of the present invention detects an analyte labeled with fluorescence or luminescence in a sample from the intensity of the fluorescence or luminescence, and comprises a non-transparent resin substrate, an immobilizing layer formed on a part of the non-transparent resin substrate, and an organic molecule immobilized on the immobilizing layer, to achieve the above-mentioned purpose.

In the biochip of the present invention, it is preferable that the organic molecule is immobilized on the immobilizing layer through at least one of silane coupling and a thiol group.

In the biochip of the present invention, it is preferable that the organic molecule is immobilized on the immobilizing layer through silane coupling.

In the biochip of the present invention, it is preferable that the immobilizing layer is a metal, a semiconductor, or an oxide.

In the biochip of the present invention, it is preferable that the immobilizing layer is silica.

In the biochip of the present invention, it is preferable for the layer of silica is formed by chemical treatment, vapor deposition or sputtering.

In the biochip of the present invention, it is preferable that the non-transparent resin substrate has a light transmittance of not more than 5% for the wavelengths of the fluorescence or luminescence of the labeled analyte.

In the biochip of the present invention, it is preferable that the non-transparent resin substrate is a resin substrate containing a non-transparent substance.

In the biochip of the present invention, it is preferable that the non-transparent substance is black in color.

In the biochip of the present invention, it is preferable that the reaction between the analyte and the organic molecule is one of a hybridization reaction and an antigen-antibody reaction.

In the biochip of the present invention, it is preferable that the biochip is further provided with one or more additional functions on the immobilizing layer, outside the area whereon the organic molecule is immobilized. It is preferable that the additional function is a hydrophobic function. Furthermore, it is preferable that the additional function is a specific adsorption function or a nonspecific adsorption prevention function.

The background fluorescence in optical detection can be reduced by the present invention. Furthermore, the substrate surface of the biochip can be made into a good quality functional surface by a simple procedure. Besides this, the fluorescence intensity at the time of optical detection can be increased by providing a plurality of additional functions to the substrate.

### BRIEF DESCRIPTION OF THE DRAWINGS

FIGS. 1A and 1B are schematic diagrams illustrating the first example of the biochip of the present invention;

FIGS. 2A and 2B are schematic diagrams illustrating the second example of the biochip of the present invention; and

FIGS. 3A and 3B are schematic diagrams illustrating the third example of the biochip of the present invention.

### DETAILED DESCRIPTION OF THE PREFERRED EMBODIMENTS

Described below are the preferred embodiments of the biochip according to the present invention.

### [Biochip]

“Biochip” here means a device in which a biomolecule, like DNA, protein, and sugar chain, or cells, etc., are immobilized on a substrate (support), the immobilized biomolecule, etc., is brought into contact with a biomolecule or any other compound, and a specific interaction that occurs is detected. “Biochip” includes DNA chips, microchips, biosensors, lab on a chip, μ-TAS (Micro Total Analysis System), etc. Non-transparent resin substrate

A resin substrate made of a thermoplastic resin, or a thermoplastic resin along with other materials, can be used as the resin substrate in the present invention. For reasons of moldability, mass-producibility, and mixability with the non-transparent substance, etc., substrates made of materials like polyethylene, polycarbonates, polymethylmethacrylate (acryl), polystyrene, polyethylene terephthalate, triacetyl cellulose, and a saturated cyclic polyolefin resin may be used. Among these materials, the use of polymethylmethacrylate or polyethylene terephthalate is particularly preferable, because of the ease of surface treatment.

The non-transparent resin substrate may be prepared by mixing a non-transparent substance in a resin substrate made from the aforesaid material. A dye, a pigment, colored beads, pellets, etc., may be used as the non-transparent substance. The background fluorescence can be substantially reduced particularly when the non-transparent substance is black in color. A pigment, especially carbon black is preferable as the black non-transparent substance because of its coloring property, heat resistance, and light absorption.

“Non-transparent” here means the non-transparent resin substrate has a light transmittance of not more than 5% for the wavelengths of the fluorescence or luminescence of the labeled analyte. The reason for specifying 5% or less is that if the light transmittance is higher than this, the absorption of light is insufficient, and the background light from the substrate or the external environment becomes non-negligible. 1% or less is preferable, and 0% is more preferable.

### [Immobilizing Layer]

As the “immobilizing layer” on the required areas of the aforesaid non-transparent resin substrate, a film of a metal, a semiconductor, or an oxide is formed, depending on the characteristics required in the biochip. The immobilizing layer formed as a film is made from a material selected from a metal, a semiconductor, or an oxide. The immobilizing layer can be made of a single material, or a combination of a metal, a semiconductor, etc. When using such combinations of materials, the film is formed according to the characteristics required for making films of different materials on different parts of the surface of the non-transparent substrate, for making the film by laminating different materials one above the other, and the like.

The immobilizing layer functions as a support whereon the organic molecule is immobilized. Furthermore, the immobilizing layer functions as an area for providing additional functions, apart from its function as the support for the immobilized organic molecule.

In the present invention, gold, silver, copper, platinum, aluminum, etc., may be used as the metal. Moreover, the metal can be a pure metal comprising a single metal element, or an alloy containing a plurality of metal elements, or metal elements and non-metal elements. In particular, it is preferable to use gold because of its reactivity with thiol groups. Methods like vapor deposition, sputtering, ion plating, electroplating, and electroless plating, and solvents like metal paste solvent, etc., may be used for making the metal film on the non-transparent resin substrate. The preferable thickness of the metal film is 1 nm to 10 μm, inclusive, and 10 to 500 nm, inclusive is more preferable.

In the present invention, a single material semiconductor represented by Si, a compound semiconductor represented by GaAs, etc., may be used as the semiconductor. Si is particularly suitable because of its reactivity with silane coupling agents. Vapor deposition, sputtering, and ion plating may be used for making the semiconductor film on the non-transparent resin substrate. The preferable thickness of the semiconductor film is 1 nm to 10 μm, inclusive, and 10 to 500 nm, inclusive is more preferable.

Furthermore, in the present invention, silica (SiO₂), TiO₂, Al₂O₃, In₂O₃ (including Sn-doped and F-doped materials), etc., can be used as the oxide. It is preferable to use SiO₂ because of its reactivity with silane coupling agents. Sputtering, vapor deposition, ion plating, and chemical modification using polysilazane solution and the like may be used for making the oxide film on the non-transparent resin substrate. The preferable thickness of the oxide film is 1 nm to 10 μm, inclusive, and 10 to 500 nm, inclusive is more preferable.

If the metal, semiconductor, or oxide is a material that cannot be easily formed into a film directly on the resin substrate, the use of a substrate having a surface layer of composition different from its interior composition can facilitate easy film formation on the substrate surface. In other words, if an intermediate layer of material to high affinity to both the substrate and the immobilizing layer is formed as a film, the immobilizing layer film can be easily formed on the resin substrate.

There are no specific restrictions on the material of the intermediate layer. It can be chosen, depending on the affinity to the substrate and to the immobilizing layer, and the method of forming the film. For example, creating a thin acryl layer or PET layer on the surface of the substrate can firmly fix the SiO₂ film when the film is formed using a polysilazane solution.

### [Immobilizing the Organic Molecule on the Immobilizing Layer]

The earlier-mentioned type of organic molecule is immobilized on the immobilizing layer through at least one of silane coupling and a thiol group.

The immobilization of the organic molecule by silane coupling may be done using a silane coupling agent. “Silane coupling agent” here means an organic silicon compound having a hydrolyzable group with affinity to the surface of the immobilizing layer, and a functional group, like a vinyl group, an epoxy group, an amino group, and a mercapto group, having affinity to the organic material.

For example, a silane coupling agent having, at one end of its molecule, a functional group (ethoxy group, etc.) that provides a silanol group (Si—OH) as the hydrolyzable group firmly binds, through its silanol group, to the surface of the immobilizing layer having OH groups, and thus can bind the organic molecule to the immobilizing layer. The silane coupling agent can be suitably adjusted, depending on the type of functional group that is to be introduced and the type of immobilizing layer involved. Silane coupling agents like aminosilane, and PEG (polyethylene glycol)-silane, can be used, depending on the type of functional group.

The organic molecule can be immobilized on the immobilizing layer, such as metal surface, through thiol groups. In the present invention, the organic molecule can be immobilized through, for instance, a compound having at one end the organic molecule to be immobilized on the immobilized layer, such as an amino group, a carboxyl group and PEG, and having a thiol group as the metal-binding functional group at the other end. Alternatively, the immobilization of the organic molecule on the immobilizing layer can be done through a single-stranded DNA or RNA, etc., having a thiol group at its end.

For instance, an aminoalkane thiol, carboxyalkane thiol, or PEG thiol may be used as the compound having a thiol group and the organic molecule attached to it.

It is also possible to immobilize the organic molecule on the immobilizing layer through one of silane coupling and a thiol group, and to further immobilize or bond another organic molecule on or to the already immobilized organic molecule through the same type or a different type of silane coupling or a thiol group.

### [Providing Additional Function(s) to the Immobilizing Layer]

The immobilizing layer of the present invention is formed as a film in order to immobilize an organic molecule on the non-transparent resin substrate by a simple method. In another aspect of the present invention, it is possible to improve the functioning of the biochip by providing additional function(s) to the immobilizing layer.

For example, a non-specific adsorption prevention function can be provided as an additional function on an area other than the detection area of the immobilizing layer, by using a silane coupling agent.

The following method can be cited as an example of providing the non-specific adsorption prevention function. A detection area is formed on the immobilizing layer by treating the surface of the non-transparent resin substrate with a silane coupling agent having one end that specifically adsorbs amino groups, or proteins such as antibodies. The surfaces of the non-transparent resin substrate other than this detection area, such as in the sample channel, are surface-treated with a silane coupling agent having at its end polyethylene glycol (PEG), etc., which prevents nonspecific adsorption of protein. Thus, areas with different functions can be formed on the immobilizing layer.

In another embodiment of the present invention, a hydrophobic function can be provided to specific areas on the immobilizing layer. The following method can be mentioned as an example thereof: coating with a solvent the main component of which is a fluorine resin, silicone resin, etc.

### [Detection of the Analyte]

The analyte is detected by one of a hybridization reaction and an antigen-antibody reaction, or a combination of these reactions.

“Hybridization reaction” here means hybridization of nucleic acid, or formation of a hybrid nucleic acid molecule. This reaction is used as a method for studying the primary structure of a nucleic acid, i.e., the homology of the base sequence, and for detecting nucleic acids with homologous base sequence. To be more specific, a target gene can be detected by exploiting the property of nucleic acids by which two single-stranded nucleic acids form hydrogen bonds only between the complimentary base pairs A-T and G-C, creating a double-spiral double-stranded nucleic acid.

The “antigen-antibody reaction” means a reaction where an antibody binds to the corresponding substance (the antigen). The antigen level in the sample can be determined by measuring, by some method, the amount of the target antigen from the amount of antibody bound to it.

The present invention is related to a biochip that can be made inexpensively, and in which the functionalizing surface treatment is simple, to be used for fluorometric detection of components in a sample.

### EXAMPLES

The present invention is described below, citing some examples. However, the scope of the present invention is not limited to these examples.

### Example 1

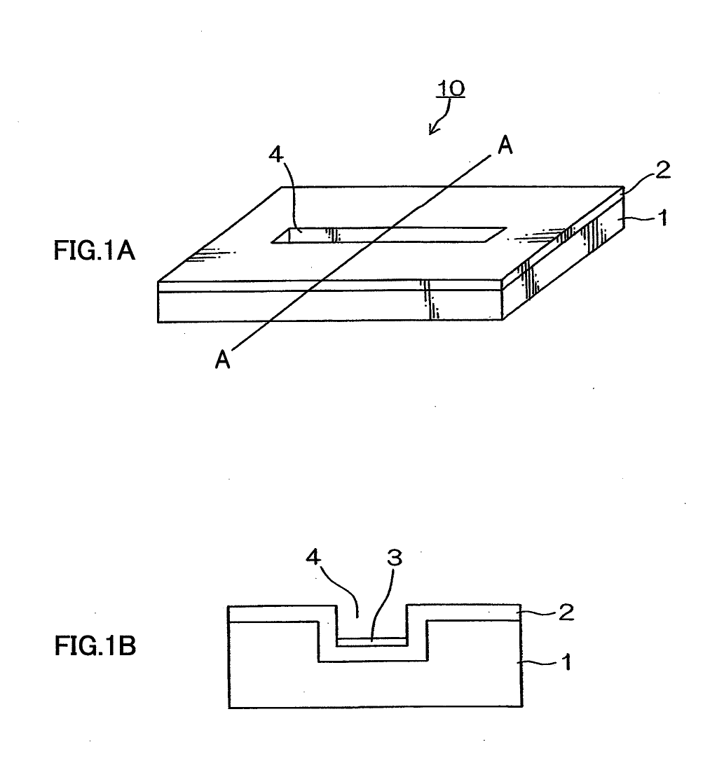

A biochip 10 of a first example of the present invention has, as illustrated in FIG. 1A, a resin substrate 1 colored to make it non-transparent (black), a micrometer scale groove 4 formed on the surface of the resin substrate 1, and a silica film 2 formed on the resin substrate 1.

The resin substrate colored (black) to make it non-transparent is prepared by adding to acryl 0.1% of black resin, as the non-transparent substance. The light transmittance of the resin substrate 1 is 0% and its thickness was 1 mm. The groove 4 of width 100 μm and depth 100 μm is formed on the resin substrate 1 using known mechanical processing technology.

The silica film 2 of thickness 300 nm is formed by chemical modification (polysilazane) over the surface of the resin substrate 1 whereon the groove 4 was formed.

An organic molecule is immobilized on the formed silica film 2 as illustrated in FIG. 1B, the cross sectional diagram at A-A, by chemically treating the surface of the silica film 2, using, for instance, a silane coupling technique. A functional area 3 (with, for instance, specific adsorption property for a biomolecule, and a property of preventing nonspecific adsorption of biomolecules) can be formed on the surface of the silica film 2 by immobilizing the organic molecule. In other words, even with a resin substrate with low activity, the formation of the silica film on it enables the application of a surface modification treatment such as silane coupling. By using this method, not only can the biochip be made inexpensively through a simple procedure, but the noise level can also be brought down because of reduced background fluorescence, and the signal can be enhanced by adsorption of the analyte exclusively in the detection area, all of which enables fluorometric detection with high sensitivity.

### Example 2

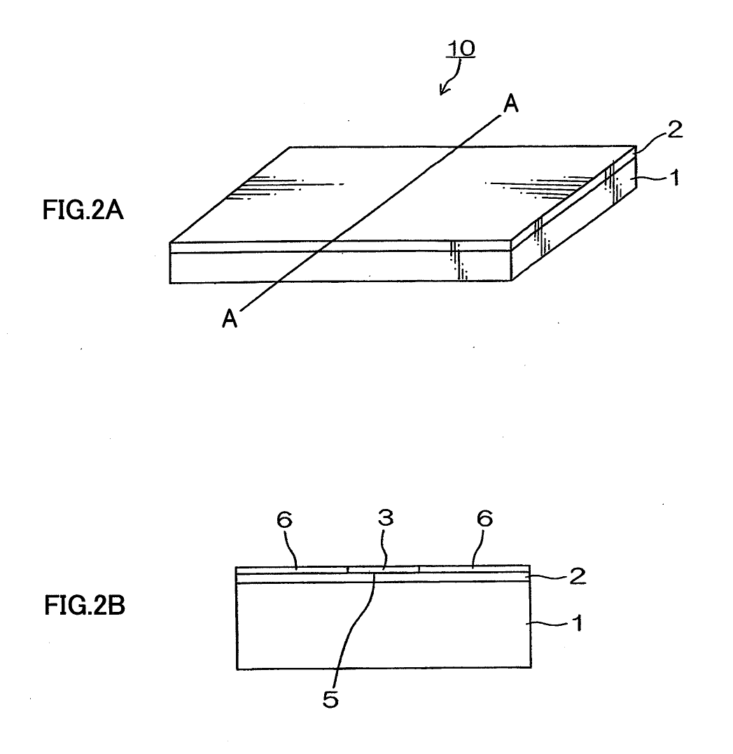

A biochip 10 of a second example of the present invention has, as illustrated in FIG. 2A, a resin substrate 1 colored to make it non-transparent (black), and a silica film 2 formed on the resin substrate 1.

The resin substrate 1 colored (black) to make it non-transparent is prepared by the same method as used in Example 1. The silica film 2 of thickness 300 nm is formed by chemical modification on the surface of the resin substrate 1, but no groove is formed on the surface of the resin substrate 1 in this example.

The area of the formed silica film 2 other than a sample path 5 is coated, for example, with a fluorine resin for hydrophobic modification, and a hydrophobic functional area 6 is formed. The hydrophobic-hydrophilic balance thus created enables a sample to flow over only the sample path 5. Then, an organic molecule is immobilized on the sample path 5 of the formed silica film 2 by chemically treating the surface of the silica film 2, using a silane coupling technology, for example. By immobilizing the organic molecule, a functional area 3 (with the property of specific adsorption of a biomolecule and the property of preventing nonspecific adsorption) can be formed on the surface of the silica film 2.

As in Example 1, even with a resin substrate having low activity, the formation of the silica film on it enables the application of a surface modification treatment such as silane coupling. By using the aforesaid method, not only can the biochip be made inexpensively through a simple procedure, but the noise level can be brought down because of the reduced background fluorescence, and the signal can be enhanced by adsorption of the analyte exclusively in the detection area, all of which enables fluorometric detection with high sensitivity.

Another aspect is that, the hydrophobic functional area 6 could be provided as another functional area at a place other than the functional area 3 on the silica film 2. This can further improve the functioning of the biochip.

### Example 3

A specific method of using the present invention will now be described, taking the application of an antigen-antibody reaction as an example.

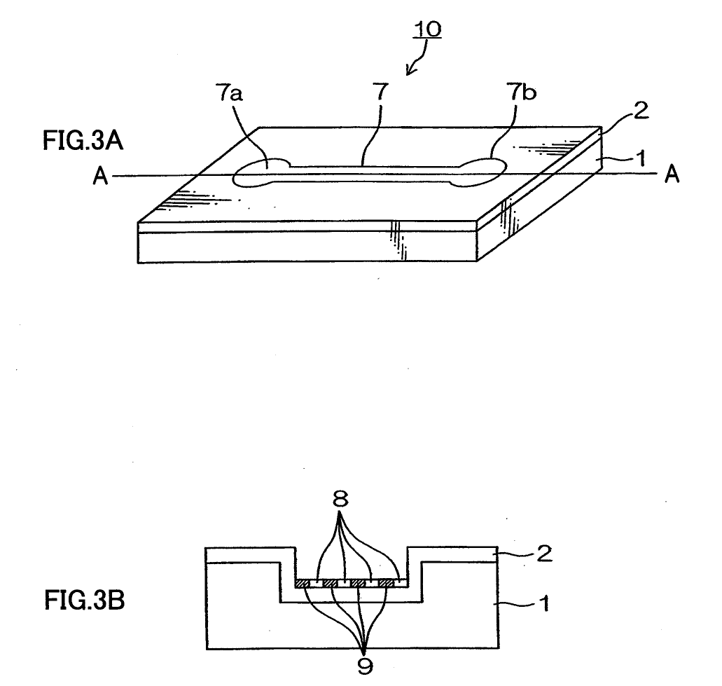

As illustrated in FIG. 3A, a biochip 10 of a third example of the present invention has a resin substrate 1 colored to make it non-transparent (black), a micrometer scale groove 7 formed on the surface of the resin substrate 1 as a channel for the sample liquid, and a silica film 2 formed on the resin substrate 1. Further, the groove 7 has an inlet 7a and an outlet 7b. The groove 7, the inlet 7a and the outlet 7b are formed using known mechanical processing technology so that the groove 7 has width 100 μm and depth 100 μm, and each of the inlet 7a and the outlet 7b has diameter 1000 μm and depth 100 μm. The silica film 2 on the surface of the resin substrate 1 is formed by treating the resin substrate 1 with a polysilazane solution.

As shown in FIG. 3B, the A-A cross-sectional diagram, a bottom surface of the groove 7, which was formed on the resin substrate 1 to serve as a channel for the sample liquid, is functionally modified in such a way that the antibody is captured only in the detection area, and nonspecific adsorption on other areas is prevented. For this modification, the area to be made into the detection area is first treated with a silane coupling agent having one end that specifically adsorbs an amino group or protein, such as the antibody, to form specific adsorption functional areas 8 in the detection area. Then, sample channels in areas other than the detection area are treated with a silane coupling agent having at its end polyethylene glycol (PEG), etc., that can prevent nonspecific adsorption of proteins, to form nonspecific adsorption prevention functional areas 9 in the sample channels. The formation of such specific adsorption functional areas 8 and nonspecific adsorption prevention functional areas 9 is not restricted to the sequence and methods used in this example.

By passing the fluorescence labeled sample liquid through a biochip treated in this manner, the analyte can be specifically adsorbed in the detection areas only. The intensity of the light is enhanced and at the same time the background fluorescence originating from the substrate, the shape of the substrate and the outside environment can be suppressed. Thus, biochips that are capable of detection with high sensitivity can be made at a low cost.

What is claimed is:

1. A biochip which detects an analyte labeled with fluorescence or luminescence in a sample, from the intensity of the fluorescence or luminescence, the biochip comprising:
a non-transparent resin substrate;
an immobilizing layer formed on a part of the non-transparent resin substrate; and
an organic molecule immobilized on the immobilizing layer.

2. The biochip according to claim 1, wherein the organic molecule is immobilized on the immobilizing layer through at least one of silane coupling and a thiol group.

3. The biochip according to claim 1, wherein the organic molecule is immobilized on the immobilizing layer through silane coupling.

4. The biochip according to claim 1, wherein the immobilizing layer is a metal, a semiconductor, or an oxide.

5. The biochip according to claim 1, wherein the immobilizing layer is silica.

6. The biochip according to claim 5, wherein the layer of silica is formed by chemical treatment, vapor deposition or sputtering.

7. The biochip according to claim 1, wherein the non-transparent resin substrate has a light transmittance of not more than 5% for the wavelengths of the fluorescence or luminescence of the labeled analyte.

8. The biochip according to claim 1, wherein the non-transparent resin substrate is a resin substrate containing a non-transparent substance.

9. The biochip according to claim 8, wherein the non-transparent substance is black in color.

10. The biochip according to claim 1, wherein the analyte is detected through one of a hybridization reaction and an antigen-antibody reaction.

11. The biochip according to claim 1, further provided with one or more additional functions on the immobilizing layer, outside the area whereon the organic molecule is immobilized.

12. The biochip according to claim 11, wherein one of the additional functions is a hydrophobic function.

13. The biochip according to claim 11, wherein one of the additional functions is a specific adsorption function.

14. The biochip according to claim 11, wherein one of the additional functions is a nonspecific adsorption prevention function.
